(19) United States
(12) Patent Application Publication
(10) Pub. No.: US 2020/0054303 A1
(43) Pub. Date: Feb. 20, 2020

(54) STETHOCOVER

(71) Applicant: Poonam Sood, San Diego, CA (US)

(72) Inventor: Poonam Sood, San Diego, CA (US)

(21) Appl. No.: 16/104,032

(22) Filed: Aug. 16, 2018

(51) Int. Cl. A61B 7/02; A61B 46/10

(52) U.S. Cl. CPC A61B 7/02; A61B 46/10

(57) ABSTRACT

A disposable stethoscope cover for the diaphragm or bell of a stethoscope to prevent the spread of microorganisms among patients. The stethoscope cover having a first surface and a second surface is made of a barrier material that is impermeable to microorganisms but capable of allowing sound to pass through. The first surface has an adhesive layer and the second surface has an antimicrobial substance, the first surface being in contact with the diaphragm or bell of the stethoscope.

### CROSS-REFERENCE TO RELATED APPLICATION

This U.S. non-provisional patent application claims the benefit of U.S. Provisional Application No. 62/547,265 tiled “Stethocover,” filed on Aug. 18, 2017, the disclosure of which is hereby incorporated by reference in its entirety.

### BACKGROUND OF THE INVENTION

The present disclosure relates to the field of stethoscope covers and, more particularly, to a disposable stethoscope cover for the diaphragm or bell of a stethoscope.

It is well known that nosocomial infections are a problem affecting the quality of health care and the leading cause of morbidity, mortality, and increased health care cost. Stethoscopes are essential tools for physicians to diagnose multiple diseases. However, they are a source of microorganisms that cause nosocomial infections. Spread of these microorganisms can lead to nosocomial outbreaks. Once the spread of antibiotic resistant microorganisms gets severe, it becomes harder to treat resulting in increased health care costs. It is estimated that the current costs per year for treating nosocomial infections in the United States is about 9.8 billion dollars. Current Medicare policies penalize hospitals if patients acquire certain conditions during their in-patient stay. Therefore, hospitals have a need to find ways to reduce health care associated infections.

It is well known that such nosocomial infections may spread from one patient to another by reusing various medical equipment. In fact, a stethoscope is a prime example of this. To prevent the spread of microorganisms, some doctors clean the diaphragm or bell of the stethoscope by wiping it with alcohol after seeing each patient. However, most of the time physicians don't take any action between patients and the same stethoscope is applied to each patient throughout the day, regardless of the etiology of their medical problem. The reason physicians don't take action is that they don't have time in their busy day, as doing even this small task, takes away critical time from seeing patients in both the inpatient and outpatient setting.

In the hospital care setting, health care providers, nurses, and health care personnel often use disposable plastic stethoscopes while seeing patients in isolated rooms in the inpatient setting to prevent the spread of microorganisms. However, it is often difficult to hear through the plastic stethoscopes, and thus an incorrect diagnosis can be made or a particular diagnosis may be missed. Also, it is costly to purchase these plastic stethoscopes as they need to be replaced after each use. Often, these plastic stethoscopes are not easily accessible as they may not be found on every floor of the hospital facility, thereby potentially delaying patient care.

In an outpatient setting, a doctor may spread microorganisms from patient to patient by using his/her personal stethoscope. Numerous well-known studies have shown that high percentages of stethoscopes are contaminated with microorganisms, mostly gram-positive organisms. Accordingly, there's a need to help reduce the spread of microorganisms thereby enhancing patient safety and care. Also, the use of health care personnel's, physicians', or nurses' personal state of the art stethoscopes is likely to reduce the costs incurred by hospitals in the purchase of disposable plastic stethoscopes.

Stethoscope sleeves, covers and methods of making the same are described, for example, in U.S. Pat. Nos. 5,365,023; 5,428,193; 5,813,992; 5,921,941; 5,949,032; 6,019,187; 6,132,086; 6,467,568; 6,520,281; 7,424,929; 7,614,477; 7.757.807; 7,614,477; 7,823,690; and 8,042,646. In general, these stethoscope sleeves and covers do not address the problems mentioned earlier or they are cumbersome to use, and the stethoscopes still require cleaning.

Accordingly, the need exists for a disposable stethoscope cover that prevents the spread of microorganisms from patient to patient thereby reducing recurrent hospital admissions and allowing quick discharge of inpatients.

### SUMMARY OF THE INVENTION

Embodiments of the inventive concepts may provide a disposable cover for stethoscopes, and methods of providing these disposable stethoscope covers.

In certain embodiments, a disposable stethoscope cover may be made of a barrier material that is impermeable to microorganisms, with an adhesive layer on a first surface. The stethoscope cover is substantially circular in shape with a protrusion extending from a portion of the circumference.

In certain embodiments, the stethoscope cover may be made in different diameter sizes to fit the varying sizes of diaphragm and bell of adult and pediatric stethoscopes. The diameter of the stethoscope cover is preferably oversized by approximately 10-20 percent in relation to the diaphragm or the bell over which the stethoscope cover will be placed.

In certain embodiments, a method includes: peeling a stethoscope cover to expose a first surface of the stethoscope cover; and engaging a diaphragm or bell of the stethoscope with the first surface of the stethoscope cover, the first surface having an adhesive layer for removably attaching the stethoscope cover to the diaphragm or the bell.

In certain embodiments, a dispensing device for storage and dispensing of the stethoscope cover may have a box or like container for storing a plurality of sealed packs, each sealed pack containing a stethoscope cover. The container has an upper side and a lower side and the upper side preferably has a dispensing opening of dimensions to allow dispensing of the packs. The container may hold a hundred stethoscope covers and may be available in rolls of hundred. The dispensing device may be placed on top of a table for easy access by a physician.

In certain embodiments, a dispensing device for storage and dispensing of the stethoscope cover may have a box or like container for storing a plurality of sealed packs, each sealed pack containing a stethoscope cover. The container preferably has a dispensing opening of dimensions to allow dispensing of the packs. The dispensing device may include a mounting plate, a cover secured to the mounting plate, and a lever. The mounting plate is adapted to be secured to a wall by a plurality of wall mounting means. The lever provided at one end of the dispensing device may be used to force a single sealed pack to be dispensed through the dispensing opening.

Other features and advantages of the inventive concepts will become apparent in view of the detailed description and the accompanying drawings.

### BRIEF DESCRIPTION OF THE DRAWINGS

FIG. 1 illustrates a stethoscope in which stethoscope covers according to example embodiments of the inventive concepts may be implemented;

FIG. 2 is a schematic diagram of a stethoscope cover according to example embodiments of the inventive concepts;

FIGS. 3A-3B illustrate stethoscope covers according to example embodiments of the inventive concepts;

FIGS. 4A-4B illustrate a stethoscope cover placed over a diaphragm of a stethoscope according to example embodiments of the inventive concepts;

FIG. 5 illustrates a stethoscope cover placed over a diaphragm of a stethoscope being removed according to example embodiments of the inventive concepts;

FIG. 6 is a schematic diagram of a device for dispensing the stethoscope covers according to example embodiments of the inventive concepts; and

FIG. 7 is a schematic diagram of a device for dispensing the stethoscope covers according to example embodiments of the inventive concepts.

### DETAILED DESCRIPTION OF THE PREFERRED EMBODIMENTS

Disclosed embodiments relate to methods for finding expertise in a given field and systems for implementing the methods.

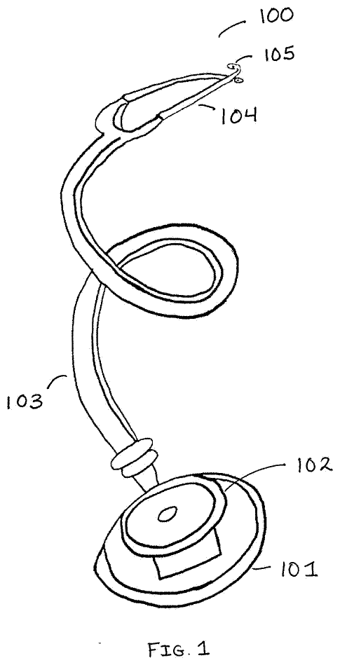

Referring now to the drawings, where like elements are designated by like reference numerals, FIG. 1 illustrates a stethoscope 100 that allows a doctor, nurse, or health care personnel to listen to a patient's heartbeat, lungs and other internal sounds. The stethoscope 100 includes a head having a diaphragm 101 and a bell 102, a tubing 103, and a headset containing ear tubes 104 and ear tips 105. Alternatively, the stethoscope may be a vented diaphragm type which does not include a cone-shaped bell but a flat-faced portion.

When the diaphragm 101 is placed against a patient's chest, back or stomach, the internal sounds of the patient vibrate the diaphragm, creating acoustic pressure waves which travels through the tubing 103 to the ear tubes 104 and onto the listener's ears. The bell 102 may at times be placed on the patient instead of the diaphragm 101. The bell 102 transmits low frequency sounds such as heart murmurs while the diaphragm 101 picks up high frequency sounds such as breath and heartbeats.

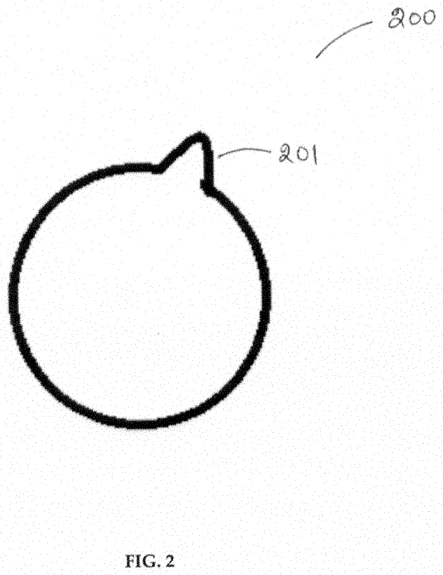

In an embodiment of the inventive concepts, as shown in FIG. 2, the stethoscope cover 200 preferably is a flexible, thin and substantially circular in shape with a protrusion 201 extending from a portion of the circumference. The circular shape of the stethoscope cover 200 is configured to correspond to a cross-section of the diaphragm 101 or the bell 102. The diameter of the stethoscope cover is preferably oversized by approximately 10-20 percent in relation to the diaphragm or the bell over which the stethoscope cover 200 will be placed so that the diaphragm or the bell does not contact the patient while in use.

For example, diaphragm diameters of adult stethoscopes may range from about 44 mm to about 51 mm, and the bell diameters may range from about 27 mm to about 32 mm. Pediatric stethoscopes, on the other hand, may have diaphragm diameters from about 27 mm to about 33 mm and bell diameters from about 19 mm to about 25 mm. The stethoscope cover 200, therefore, may be made in different diameter sizes to fit the diaphragm and bell of adult and pediatric stethoscopes. Accordingly, the diameter of the stethoscope cover 200 may be of varying sizes or a single size. For example, the largest diameter of a diaphragm of a Littmann stethoscope is about 51 mm and therefore, one set of stethoscope covers may have a diameter of about 56 mm to include a 5 mm oversize margin. Another set of stethoscope covers may have a diameter of about 38 mm including a 5 mm oversize margin. Thus, the diameter of the stethoscope cover 200 may be in the range of about 20 mm to about 58 mm.

The stethoscope cover 200 is preferably thin and may vary in thickness. For example, the thickness of the stethoscope cover 200 may be about 1.2 Mil to about 3.5 Mil where 1 Mil is 1/1000 inch.

The protrusion 201 of the stethoscope cover 200 helps in easy removal of the stethoscope cover 200 after use and the protrusion may be color coded. The protrusion 201 of the stethoscope cover 200 may be of varying sizes based on the diameter of the stethoscope cover. For example, a stethoscope cover having a diameter of about 51 mm may have a protrusion of about 15 mm. The protrusion may be in the range of about 8 mm to about 15 mm.

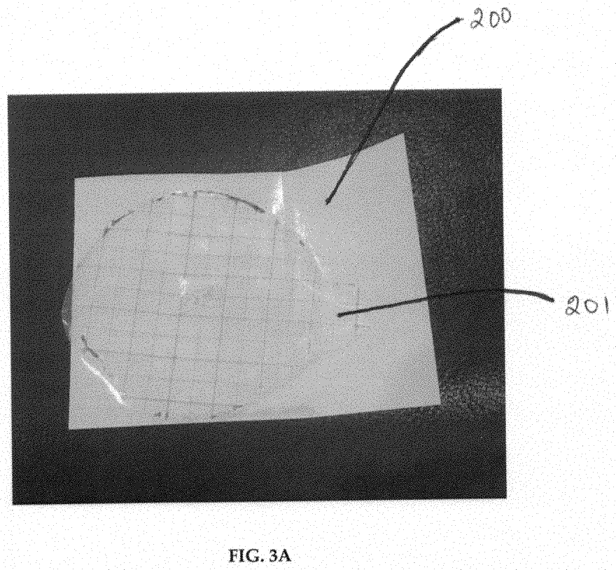

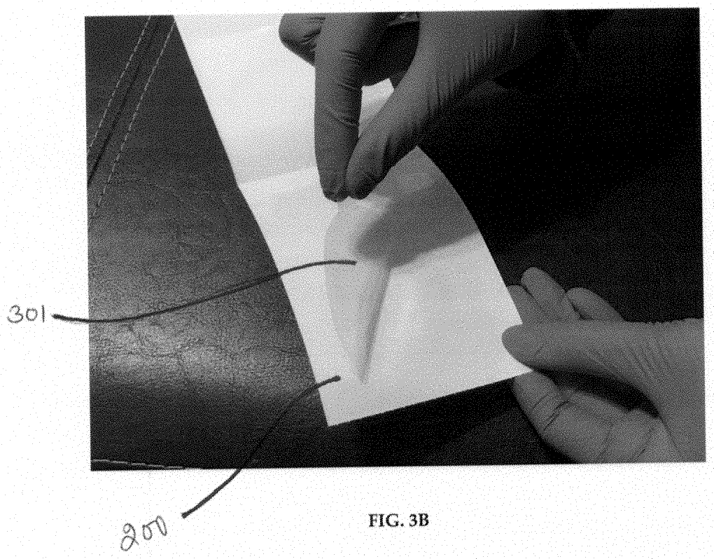

In an embodiment of the inventive concepts, as shown in FIGS. 3A-3B, the stethoscope cover 200 is preferably formed of a thin polyurethane membrane with a first surface 301 coated with a layer of anacrylic adhesive, the polyurethane membrane being permeable to both water vapor and oxygen but impermeable to micro-organisms. The stethoscope cover 200 may be made of, for example, bi-axially oriented polypropylene which is 10993 compliant.

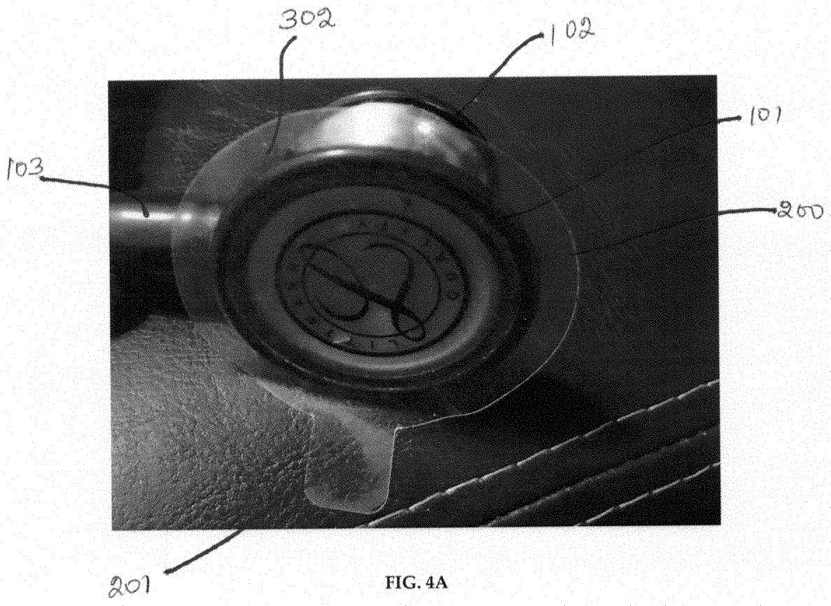

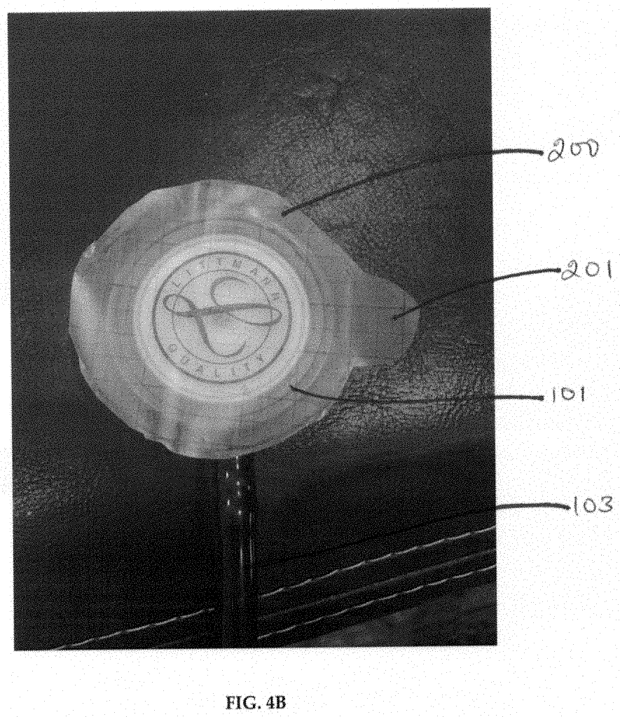

In other embodiments of the inventive concepts, the stethoscope cover 200 may be formed of other materials including cellophane, acetate, latex rubber, synthetic rubber, vinyl, or polypropylene. The stethoscope cover 200 may be coated with any anti-bacterial or anti-microbial material on a second surface 302 (FIG. 4A) and an adhesive layer on a first surface 301. The adhesive layer may be made from emulsion acrylic, solvent acrylic, or the like. The antimicrobial material may be, for example, potassium sorbate, Sorbic acid, Nisin, Carvacrol, Linalool, Chitosan, Lauric acid ester, or Cinnamaldehyde.

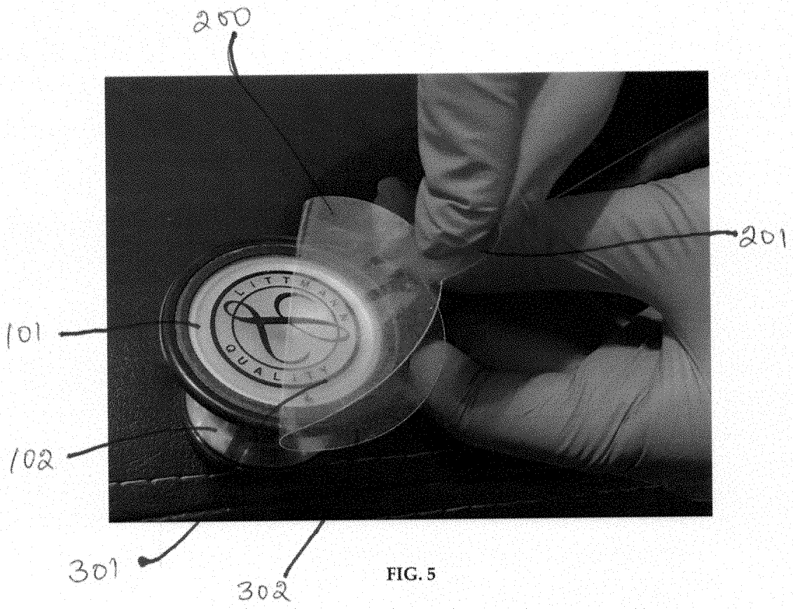

In an embodiment of the inventive concepts, as shown in FIGS. 4-5, the stethoscope cover 200 is placed on the diaphragm 101 of a stethoscope. The adhesive layer on a first surface 301 of the stethoscope cover 200 attaches to the diaphragm 101 of the stethoscope. The stethoscope 100 is then used with the barrier material making contact with a patient's body. Subsequent to use, the health care personnel removes the stethoscope cover 200 by holding the protrusion 201 and pulling it to peal the stethoscope cover 200 from the diaphragm 101 or bell 102 and to discard the stethoscope cover 200 thereafter.

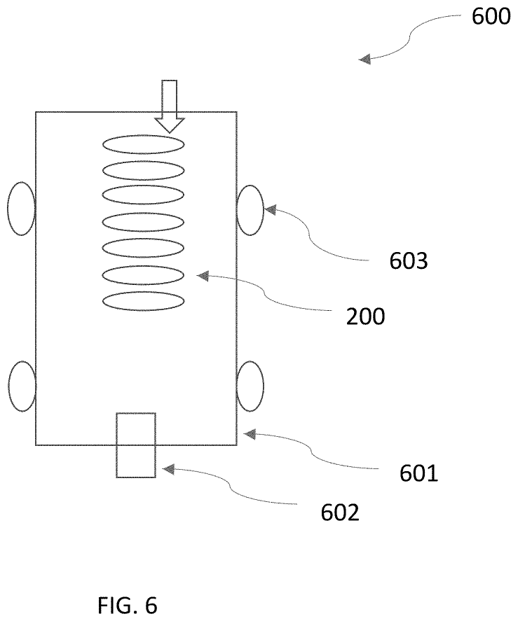

In an embodiment of the inventive concepts, as shown in FIG. 6, a dispensing device 600 for the stethoscope covers 200, as described herein, may be a wall mounted, stand-alone or portable device. The dispensing device has a mounting plate, a container 601, a cover secured to the mounting plate, a lever 602 and a viewing window. The mounting plate is adapted to be secured to a wall by a plurality of wall mounting means 603. The wall mounting means may be, for example, suction cups or Velcro.

The box or like container 601 is adapted to store a plurality of sealed packs, each sealed pack containing a stethoscope cover 200. The lever 602 provided at one end of the dispensing device 600 forces a single sealed pack to be dispensed through a dispensing opening dimensioned to allow dispensing of the packs.

The dispensing device 600 may preferably be mounted on the wall outside or inside of each patient room and preferably next to a hand sanitizing solution. In this manner, health care personnel may clean his/her hands and obtain a stethoscope cover 200 prior to entering a patient room or prior to examining a patient in a patient room.

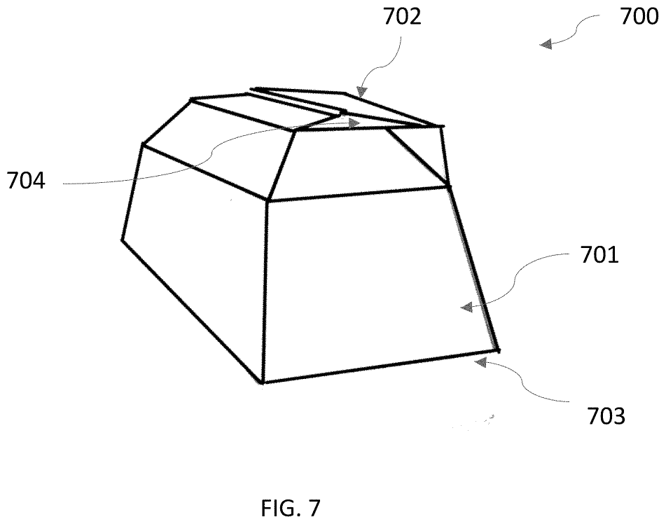

In an embodiment of the inventive concepts, as shown in FIG. 7, a dispensing device 700 for the stethoscope covers 200, as described herein, may be a wall mounted, stand-alone or portable device. The box or like container 701 is adapted to store a plurality of sealed packs, each sealed pack containing a stethoscope cover 200. The box or like container 701 has an upper side 702 and a lower side 703, the upper side 702 preferably having a dispensing opening 704 of dimensions to allow dispensing of the packs. The box or like container 701 may hold a hundred stethoscope covers 200 and may be available in rolls of hundred. The dispensing device 700 may be placed on top of a table next to a sink for easy access by a physician once the physician washes their hands before seeing a patient.

While various aspects of the inventive concepts have been described herein with reference to example embodiments, it should be understood that the inventive concepts are not limited thereto. Those having ordinary skill in the art and access to the teachings provided herein will recognize additional modifications, applications, embodiments and substitution of equivalents all fall within the scope of the inventive concepts. Therefore, it should be understood that the above embodiments are not limiting, but illustrative. The scopes of the inventive concepts are to be determined by the broadest permissible interpretation of the following claims and their equivalents. Accordingly, the inventive concepts are not to be considered as limited or restricted by the foregoing description.

What is claimed as new and desired to be protected by Letters Patent of the United States is:

1. A disposable stethoscope cover, comprising:
a first surface having an adhesive layer, the adhesive layer being in contact with a diaphragm or bell of a stethoscope and the adhesive layer removably attaching the stethoscope cover to the diaphragm or the bell;
a second surface comprising one or more of an antimicrobial and/or antiseptic material;
wherein the stethoscope cover is substantially circular in shape and having a protrusion extending from a portion of a circumference of the stethoscope cover.

2. The disposable stethoscope cover of claim 1, wherein the stethoscope cover is substantially flexible and thin.

3. The disposable stethoscope cover of claim 1, wherein diameter of the stethoscope cover is in the range of about 38 mm to 56 mm.

4. The disposable stethoscope cover of claim 1, wherein thickness of the stethoscope cover is the range of about 1.2 Mil to about 3.5 Mil.

5. The disposable stethoscope cover of claim 1, wherein the protrusion is color coded.

6. The disposable stethoscope cover of claim 1, wherein the stethoscope cover is made of material comprising polyurethane, cellophane, acetate, latex rubber, synthetic rubber, vinyl, or polypropylene.

7. The disposable stethoscope cover of claim 1, wherein the adhesive layer of the first surface may be made from emulsion acrylic, solvent acrylic, or the like.

8. A method for covering a stethoscope, comprising:
peeling a stethoscope cover to expose a first surface of the stethoscope cover; and
engaging a diaphragm or bell of the stethoscope with the first surface of the stethoscope cover, the first surface having an adhesive layer for removably attaching the stethoscope cover to the diaphragm or the bell.

9. The method as recited in claim 8, further comprising: dispensing the stethoscope cover from a container adapted to store a plurality of stethoscope covers.
